(19) United States
(12) Patent Application Publication
(10) Pub. No.: US 2006/0029953 A1
(43) Pub. Date: Feb. 9, 2006

(54) Methods and kits comprising amplification and ligation for analyzing target polynucleotide sequences

(75) Inventor: Stephen J. Sharf, Castro Valley, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/171,823

(22) Filed: Jun. 29, 2005

Related U.S. Application Data
(60) Provisional application No. 60/584,229, filed on Jun. 29, 2004.

(51) Int. Cl. C12Q 1/68; C12P 19/34

(52) U.S. Cl. 435/6; 435/91.2

(57) ABSTRACT

The present teachings relate to methods and kits for detecting one or more target polynucleotide sequences in a sample. In some embodiments of the present teachings, a probe set comprising a target identifying portion is hybridized to a strand of an amplification product and ligated together to form a ligation product. The ligation product can be queried with a mobility probe, and analysis of the mobility probe with a mobility dependent analysis technique is used to determine the target polynucleotide sequence. The present teachings can employ highly multiplexed amplification and ligation reactions to query a variety of target polynucleotide sequences, including single nucleotide polymorphisms.

### CROSS REFERENCE TO RELATED APPLICATIONS

This application claims priority benefit under 35 U.S.C. § 119(e) from U.S. Provisional Patent Application No. 60/584,229, filed Jun. 29, 2004, which is incorporated herein by reference.

### FIELD

The present teachings generally relate to methods and kits analyzing polynucleotide sequences. More specifically, the teachings relate to amplification and ligation based methods and kits for determining single nucleotide polymorphisms in target polynucleotide sequences.

### BACKGROUND

The detection of the presence or absence of (or quantity of) one or more target polynucleotides in a sample or samples containing one or more target sequences is commonly practiced. For example, the detection of cancer and many infectious diseases, such as AIDS and hepatitis, routinely includes screening biological samples for the presence or absence of diagnostic nucleic acid sequences. Also, detecting the presence or absence of nucleic acid sequences is often used in forensic science, paternity testing, genetic counseling, and organ transplantation.

An organism's genetic makeup is determined by the genes contained within the genome of that organism. Genes are composed of long strands or deoxyribonucleic acid (DNA) polymers that encode the information needed to make proteins. Properties, capabilities, and traits of an organism often are related to the types and amounts of proteins that are, or are not, being produced by that organism.

A protein can be produced from a gene as follows. First, the information that represents the DNA of the gene that encodes a protein, for example, protein “X”, is converted into ribonucleic acid (RNA) by a process known as “transcription.” During transcription, a single-stranded complementary RNA copy of the gene is made. Next, this RNA copy, referred to as protein X messenger RNA (mRNA), is used by the cell's biochemical machinery to make protein X, a process referred to as “translation.” Basically, the cell's protein manufacturing machinery binds to the mRNA, “reads” the RNA code, and “translates” it into the amino acid sequence of protein X. In summary, DNA is transcribed to make mRNA, which is translated to make proteins.

The amount of protein X that is produced by a cell often is largely dependent on the amount of protein X mRNA that is present within the cell. The amount of protein X mRNA within a cell is due, at least in part, to the degree to which gene X is expressed. Whether a particular gene or gene variant is expressed, and if so, to what level, can have a significant impact on the organism.

Further, the kind of protein X within a cell is due, at least in part, to the sequence variant that encodes the protein. One common kind of sequence variation that can affect the kind of protein cell's make is referred to as single nucleotide polymorphisms (SNPs). Whether a particular gene or gene variant is present, and if so, with how many copies, can have significant impact on an organism.

### SUMMARY

The present teachings provide methods and kits for analyzing a target polynucleotide sequence. The method comprises first amplifying a portion of the polynucleotide sequence of interest and producing an amplification product. Then mixing the amplification product with a ligation probe set, the ligation probe set comprising a first ligation probe having a target specific portion complementary to a first strand of the amplicon, and a second ligation probe having a target specific portion complementary to the first strand of the amplicon, wherein the first ligation probe comprises an identifying portion, wherein the second ligation probe comprises an affinity moiety, and wherein the ligation probes in a particular set are suitable for ligation together when hybridized adjacent to one another on the same strand of the amplification product. After hybridization of the ligation probe, the first ligation probe is ligated to the second ligation probe to produce a ligation product, wherein the ligation product comprises (a) the target identifying portion, (b) the target specific portions ligated together, and (C) the affinity moiety. Thereafter, the ligation product is contacted with an affinity moiety binder to immobilize the ligation product. The immobilized ligation products are then hybridized to a mobility probe, wherein the mobility probe comprises a sequence complementary to the target identifying portion. The mobility probe is then eluted and detected, thereby analyzing the target polynucleotide sequence. The present teachings can be applied in highly multiplexed amplification and ligation reactions, thereby allowing for the determination of a large number of target polynucleotide sequences, including the analysis of single nucleotide polymorphisms using mobility dependent analysis techniques such as capillary electrophoresis.

### BRIEF DESCRIPTION OF THE DRAWINGS

FIG. 1 depicts certain method embodiments of the present teachings.

FIG. 2 depicts certain method embodiments of the present teachings.

FIG. 3 depicts certain method embodiments of the present teachings.

### DESCRIPTION OF EXEMPLARY EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall prevail.

It is to be understood that both the foregoing general description and the following detailed description are exemplary and explanatory only and are not restrictive of the invention. In this application, the use of the singular includes the plural unless the context specifically dictates otherwise. For example, “a probe” means that more than one probe can be present; for example, one or more copies of a particular probe species, as well as one or more versions of a particular probe type. Also, the use of “or” means “and/or” unless stated otherwise. Similarly, “comprise”, “comprises”, “comprising”, “include”, “includes”, and “including” are not intended to be limiting.

Definitions

As used herein, the “probes,” “primers,” “targets,” “oligonucleotides,” “polynucleotides,” “nucleobase sequences,” and “oligomers” of the present teachings can be comprised of at least one of ribonucleotides, deoxynucleotides, modified ribonucleotides, modified deoxyribonucleotides, modified phosphate-sugar-backbone oligonucleotides, nucleotide analogs, and combinations thereof, and can be single stranded, double stranded, or contain portions of both double stranded and single stranded sequence, as appropriate.

The term “nucleotide”, as used herein, generically encompasses the following terms, which are defined below: nucleotide base, nucleoside, nucleotide analog, extendable, and universal nucleotide.

The term “nucleotide base”, as used herein, refers to a substituted or unsubstituted parent aromatic ring or rings. In some embodiments, the aromatic ring or rings contain at least one nitrogen atom. In some embodiments, the nucleotide base is capable of forming Watson-Crick and/or Hoogsteen hydrogen bonds with an appropriately complementary nucleotide base. Exemplary nucleotide bases and analogs thereof include, but are not limited to, purines such as 2-aminopurine, 2,6-diaminopurine, adenine (A), ethenoadenine, N6-Δ2-isopentenyladenine (6iA), N6-Δ2-isopentenyl-2-methylthioadenine (2 ms6iA), N6-methyladenine, guanine (G), isoguanine, N2-dimethylguanine (dmG), 7-methylguanine (7mG), 2-thiopyrimidine, 6-thioguanine (6sG) hypoxanthine and 06-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, 04-methylthymine, uracil (U), 4-thiouracil (4sU) and 5,6-dihydrouracil (dihydrouracil; D); indoles such as nitroindole and 4-methylindole; pyrroles such as nitropyrrole; nebularine; base (Y); etc. In some embodiments, nucleotide bases are universal nucleotide bases. Additional exemplary nucleotide bases can be found, e.g., in Fasman, 1989, Practical Handbook of Biochemistry and Molecular Biology, pp. 385-394, CRC Press, Boca Raton, Fla., and the references cited therein. Further examples of universal bases can be found for example in Loakes, N.A.R. 2001, vol 29:2437-2447 and Seela N.A.R. 2000, vol 28:3224-3232.

The term “nucleoside”, as used herein, refers to a compound having a nucleotide base covalently linked to the C-1′ carbon of a pentose sugar. In some embodiments, the linkage is via a heteroaromatic ring nitrogen. Typical pentose sugars include, but are not limited to, those pentoses in which one or more of the carbon atoms are each independently substituted with one or more of the same or different —R, —OR, —NRR or halogen groups, where each R is independently hydrogen, (C1-C6) alkyl or (C5-C₁₄) aryl. The pentose sugar may be saturated or unsaturated. Exemplary pentose sugars and analogs thereof include, but are not limited to, ribose, 2′-deoxyribose, 2′-(C1-C6)alkoxyribose, 2′-(C5-C14)aryloxyribose, 2′,3′-dideoxyribose, 2′,3′-didehydroribose, 2′-deoxy-3′-haloribose, 2′-deoxy-3′-fluororibose, 2′-deoxy-3′-chlororibose, 2′-deoxy-3′-α minoribose, 2′-deoxy-3′-(C1-C6)alkylribose, 2′-deoxy-3′-(C1-C6)alkoxyribose and 2′-deoxy-3′-(C5-C14)aryloxyribose. Also see e.g. 2′-O-methyl, 4′-α-anomeric nucleotides, 1′-α-anomeric nucleotides (Asseline (1991) Nucl. Acids Res. 19:4067-74), 2′-4′- and 3′-4′-linked and other “locked” or “LNA”, bicyclic sugar modifications (WO 98/22489; WO 98/39352; WO 99/14226). “LNA” or “locked nucleic acid” is a DNA analogue that is conformationally locked such that the ribose ring is constrained by a methylene linkage between the 2′-oxygen and the 3′- or 4′-carbon. The conformation restriction imposed by the linkage often increases binding affinity for complementary sequences and increases the thermal stability of such duplexes.

Exemplary LNA sugar analogs within a polynucleotide include the structures: 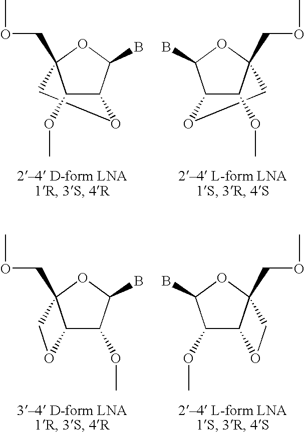
where B is any nucleobase.

Sugars include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleobase is purine, e.g. A or G, the ribose sugar is attached to the N⁹-position of the nucleobase. When the nucleobase is pyrimidine, e.g. C, T or U, the pentose sugar is attached to the N¹-position of the nucleobase (Kornberg and Baker, (1992) DNA Replication, 2^{nd} Ed., Freeman, San Francisco, Calif.).

One or more of the pentose carbons of a nucleoside may be substituted with a phosphate ester having the formula: 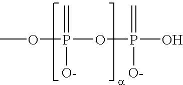
where α is an integer from 0 to 4. In some embodiments, α is 2 and the phosphate ester is attached to the 3′- or 5′-carbon of the pentose. In some embodiments, the nucleosides are those in which the nucleotide base is a purine, a 7-deazapurine, a pyrimidine, a universal nucleotide base, a specific nucleotide base, or an analog thereof.

The term “nucleotide analog”, as used herein, refers to embodiments in which the pentose sugar and/or the nucleotide base and/or one or more of the phosphate esters of a nucleoside may be replaced with its respective analog. In some embodiments, exemplary pentose sugar analogs are those described above. In some embodiments, the nucleotide analogs have a nucleotide base analog as described above. In some embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions. Other nucleic acid analogs and bases include for example intercalating nucleic acids (INAS, as described in Christensen and Pedersen, 2002), and AEGIS bases (Eragen, U.S. Pat. No. 5,432,272). Additional descriptions of various nucleic acid analogs can also be found for example in (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 1 1 0:4470 (1988); and Pauwels et al., Chemica Scripta 26:141 91986)), phosphorothioate (Mag et al., Nucleic Acids Res. 19:1437 (1991); and U.S. Pat. No. 5,644,048. Other nucleic analogs comprise phosphorodithioates (Briu et al., J. Am. Chem. Soc. 11 1:2321 (1989), 0-methylphophoroamidite linkages (see Eckstein, Oligonucleotides and Analogues: A Practical Approach, Oxford University Press), those with positive backbones (Denpcy et al., Proc. Natl. Acad. Sci. USA 92:6097 (1995); non-ionic backbones (U.S. Pat. Nos. 5,386,023, 5,386,023, 5,637,684, 5,602,240, 5,216,141, and 4,469,863. Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1 9 4): Chaq.ters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are also described in Rawls, C & E News Jun. 2, 1997 page 35.

The term “universal nucleotide base” or “universal base”, as used herein, refers to an aromatic ring moiety, which may or may not contain nitrogen atoms. In some embodiments, a universal base may be covalently attached to the C-1′ carbon of a pentose sugar to make a universal nucleotide. In some embodiments, a universal nucleotide base does not hydrogen bond specifically with another nucleotide base. In some embodiments, a universal base hydrogen bonds with a nucleotide base, up to and including all nucleotide bases in a particular target polynucleotide. In some embodiments, a nucleotide base may interact with adjacent nucleotide bases on the same nucleic acid strand by hydrophobic stacking. Universal nucleotides include, but are not limited to, deoxy-7-azaindole triphosphate (d7AITP), deoxyisocarbostyril triphosphate (dICSTP), deoxypropynylisocarbostyril triphosphate (dPICSTP), deoxymethyl-7-azaindole triphosphate (dM7AITP), deoxylmPy triphosphate (dlmPyTP), deoxyPP triphosphate (dPPTP), or deoxypropynyl-7-azaindole triphosphate (dP7AITP). Further examples of such universal bases can be found, inter alia, in Published U.S. application Ser. No. 10/290,672, and U.S. Pat. No. 6,433,134.

As used herein, the terms “polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or internucleotide analogs. Polynucleotides have associated counter ions, such as H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. Polynucleotides may be comprised of internucleotide, nucleobase and/or sugar analogs. Polynucleotides typically range in size from a few monomeric units, e.g. 3-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine, “G” denotes deoxyguanosine, and “T” denotes thymidine, unless otherwise noted.

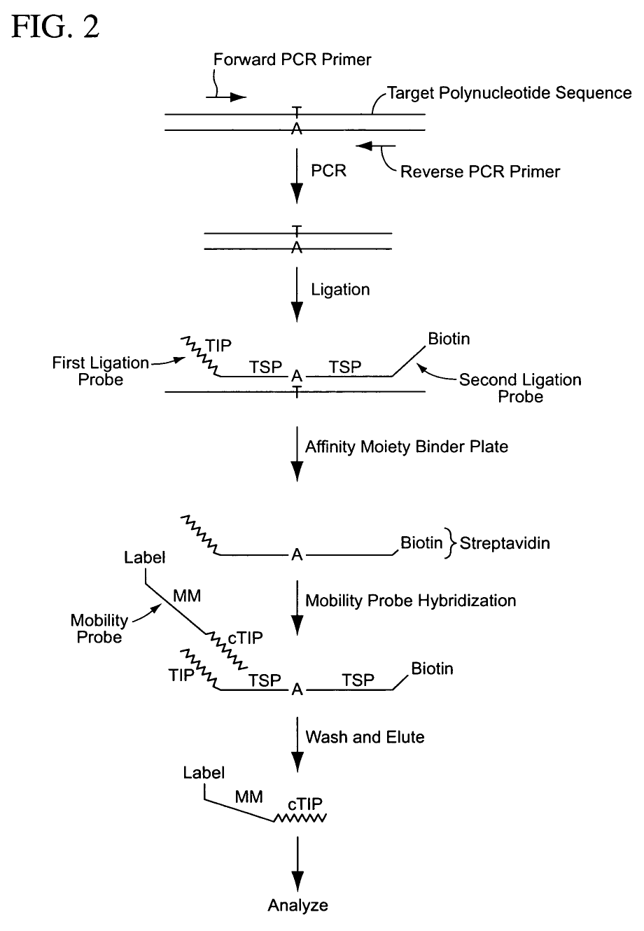

As used herein, “nucleobase” means those naturally occurring and those non-naturally occurring heterocyclic moieties commonly known to those who utilize nucleic acid technology or utilize peptide nucleic acid technology to thereby generate polymers that can sequence specifically bind to nucleic acids. Non-limiting examples of suitable nucleobases include: adenine, cytosine, guanine, thymine, uracil, 5-propynyl-uracil, 2-thio-5-propynyl-uracil, 5-methlylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobase include those nucleobases illustrated in FIGS. 2(A) and 2(B) of Buchardt et al. (WO92/20702 or WO92/20703).

As used herein, “nucleobase sequence” means any segment, or aggregate of two or more segments (e.g. the aggregate nucleobase sequence of two or more oligomer blocks), of a polymer that comprises nucleobase-containing subunits. Non-limiting examples of suitable polymers or polymers segments include oligodeoxynucleotides (e.g. DNA), oligoribonucleotides (e.g. RNA), peptide nucleic acids (PNA), PNA chimeras, PNA combination oligomers, nucleic acid analogs and/or nucleic acid mimics.

As used herein, “polynucleobase strand” means a complete single polymer strand comprising nucleobase subunits. For example, a single nucleic acid strand of a double stranded nucleic acid is a polynucleobase strand.

As used herein, “nucleic acid” is a nucleobase sequence-containing polymer, or polymer segment, having a backbone formed from nucleotides, or analogs thereof. Preferred nucleic acids are DNA and RNA.

As used herein, “peptide nucleic acid” or “PNA” means any oligomer or polymer segment (e.g. block oligomer) comprising two or more PNA subunits (residues), but not nucleic acid subunits (or analogs thereof), including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, and 6,107,470. The term PNA shall also apply to any oligomer or polymer segment comprising two or more subunits of those nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000.

In some embodiments, PNA is an oligomer or polymer segment comprising two or more covalently linked subunits of the formula found in paragraph 76 of U.S. Patent Application 2003/0077608A1 wherein, each J is the same or different and is selected from the group consisting of H, R¹, OR¹, SR¹, NHR¹, NR¹₂, F, Cl, Br and I. Each K is the same or different and is selected from the group consisting of O, S, NH and NR¹. Each R¹ is the same or different and is an alkyl group having one to five carbon atoms that may optionally contain a heteroatom or a substituted or unsubstituted aryl group. Each A is selected from the group consisting of a single bond, a group of the formula; -(CJ₂)ₛ- and a group of the formula; -(CJ₂)ₛC(O)—, wherein, J is defined above and each s is a whole number from one to five. Each t is 1 or 2 and each u is 1 or 2. Each L is the same or different and is independently selected from: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine), other naturally occurring nucleobase analogs or other non-naturally occurring nucleobases.

In some other embodiments, a PNA subunit comprises a naturally occurring or non-naturally occurring nucleobase attached to the N-α-glycine nitrogen of the N-[2-(aminoethyl)]glycine backbone through a methylene carbonyl linkage; this currently being the most commonly used form of a peptide nucleic acid subunit.

As used herein, “target polynucleotide sequence” is a nucleobase sequence of a polynucleobase strand sought to be determined. It is to be understood that the nature of the target sequence is not a limitation of this invention. The polynucleobase strand comprising the target sequence may be provided from any source. For example, the target sequence may exist as part of a nucleic acid (e.g. DNA or RNA), PNA, nucleic acid analog or other nucleic acid mimic. The target can be methylated, non-methylated, or both. The sample containing the target sequence may be from any source, and is not a limitation of the present teachings. Further, it will be appreciated that “target” can refer to both a “target polynucleotide sequence” as well as surrogates thereof, for example ligation products, amplification products, and sequences encoded therein.

As used herein, “forward” and “reverse” are used to indicate relative orientation of probes on a target, and generally refer to a 5′ to 3′ “forward” oriented primer hybridized to the 3′ end of the ‘top’ strand of a target polynucleotide, and a 5′ to 3′ “reverse” oriented primer hybridized to the 3′ end of the bottom strand of a target polynucleotide. As will be recognized by those of skill in the art, these terms are not intended to be limiting, but rather provide illustrative orientation in any given embodiment.

As used herein, the term “sample” refers to a mixture from which the at least one target polynucleotide sequence is derived, such sources including, but not limited to, raw viruses, prokaryotes, protists, eukaryotes, plants, fungi, and animals. These sample sources may include, but are not limited to, whole blood, a tissue biopsy, lymph, bone marrow, amniotic fluid, hair, skin, semen, biowarfare agents, anal secretions, vaginal secretions, perspiration, various environmental samples (for example, agricultural, water, and soil), research samples generally, purified samples generally, and cultured cells. It will be appreciated that nucleic acids can be isolated from samples using any of a variety of procedures known in the art, for example the Applied Biosystems ABI Prism TM 6100 Nucleic Acid PrepStation, and the ABI Prism TM 6700 Automated Nucleic Acid Workstation, Boom et al., U.S. Pat. No. 5,234,809, etc. It will be appreciated that nucleic acids can be cut or sheared prior to analysis, including the use of such procedures as mechanical force, sonication, restriction endonuclease cleavage, or any method known in the art.

It will be appreciated that the selection of the probes to query a given target polynucleotide sequence, and the selection of which target polynucleotide sequences to collect in a given reaction, will involve procedures generally known in the art, and can involve the use of algorithms to select for those sequences with minimal secondary and tertiary structure, those targets with minimal sequence redundancy with other regions of the genome, those target regions with desirable thermodynamic characteristics, and other parameters desirable for the context at hand. In some embodiments, probes can further comprise various modifications such as a minor groove binder (see for example U.S. Pat. No. 6,486,308) to further provide desirable thermodynamic characteristics.

As used herein, the term “probe set” refers to at least one first probe and at least one second probe that together query a given target polynucleotide sequence.

As used herein, the term “first probe” refers generally to at least one oligonucleotide that can hybridize to a target polynucleotide sequence adjacent to a second probe, and that generally comprises a target specific portion, wherein the target specific portion comprises a discriminating region, and a target identifying portion. When querying a particular SNP locus for example, a probe set can comprise a “first probe one” and a “first probe two,” and potentially more first probes, all of which differ in their discriminating regions and in their target identifying portions. When querying a plurality of SNP loci in a given reaction, for example, a plurality of probe sets can be employed, wherein the first probes of all the probe sets differ in their target specific portion corresponding to the SNP locus of interest, as well as differ in their target identifying portions.

As used herein, the term “second probe” refers generally to at least one oligonucleotide that can hybridize to a target polynucleotide sequence adjacent to at least one first probe, and that generally comprises a target specific portion and an affinity moiety.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “target specific portion” refers to the portion of a probe substantially complementary to a target polynucleotide sequence, and can further comprise a discriminating region.

The term “corresponding” as used herein refers to at least one specific relationship between the elements to which the term refers. For example, at least one first probe of a probe set corresponds to at least one second probe of the same probe set, and vice versa. At least one primer is designed to anneal with the primer portion of at least one corresponding probe, at least one corresponding ligation product, at least one corresponding amplified ligation product, or combinations thereof. The target-specific portions of the probes of a particular probe set can be designed to hybridize with a complementary or substantially complementary region of the corresponding target polynucleotide sequence. A particular affinity moiety can bind to the corresponding affinity moiety binder, for example but not limited to, the affinity moiety binder streptavidin binding to the affinity moiety biotin. A particular mobility probe can hybridize with the corresponding identifier portion or identifying portion complement. A particular discriminating region can hybridize to the corresponding nucleotide or nucleotides on the target polynucleotide, as so forth.

As used herein the term “contiguous” refers to the absence of a gap between the terminal nucleobase of at least two adjacently hybridized oligonucleotides, such that the at least two oligonucleotides are abutting one another and are potentially suitable for ligation. Adjacent refers to contiguous, as well as non-contiguous hybridization of two oligonucleotides.

As used herein, the term “discriminating region” refers generally to that region of the target specific portion of a first probe that can, or cannot, be complementary with a corresponding region of the target polynucleotide sequence. In some embodiments, the discriminating nucleotide is located at the 3′ end of the target specific portion of a first probe, though it will be appreciated that the discriminating region can be in other regions of the first probe as well. It will be appreciated that the discriminating region can refer to a single nucleotide, or more than one single nucleotide. In general, the discriminating region allows a first ligation probe to hybridize to a given allele in a base-specific manner.

As used herein the terms “annealing” and “hybridization” are used interchangeably and mean the complementary base-pairing interaction of one nucleic acid with another nucleic acid that results in formation of a duplex, triplex, or other higher-ordered structure. In some embodiments, the primary interaction is base specific, e.g., A/T and G/C, by Watson/Crick and Hoogsteen-type hydrogen bonding. In some embodiments, base-stacking and hydrophobic interactions may also contribute to duplex stability. Conditions for hybridizing nucleic acid probes and primers to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the probes and the complementary target sequences, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. Further, in general probes and primers of the present teachings are designed to be complementary to a target sequence, such that hybridization of the target and the probes or primers occurs. It will be appreciated, however, that this complementarity need not be perfect; there can be any number of base pair mismatches that will interfere with hybridization between the target sequence and the single stranded nucleic acids of the present teachings. However, if the number of base pair mismatches is so great that no hybridization can occur under even the least stringent of hybridization conditions, the sequence is not a complementary target sequence. Thus, by “substantially complementary” herein is meant that the probes or primers are sufficiently complementary to the target sequence to hybridize under the selected reaction conditions.

As used herein, the terms “label” refers to detectable moieties that can be attached to an oligonucleotide, mobility probe, or otherwise be used in a reporter system, to thereby render the molecule detectable by an instrument or method. For example, a label can be any moiety that: (i) provides a detectable signal; (ii) interacts with a second label to modify the detectable signal provided by the first or second label; or (iii) confers a capture function, e.g. hydrophobic affinity, antibody/antigen, ionic complexation. The skilled artisan will appreciate that many different species of reporter labels can be used in the present teachings, either individually or in combination with one or more different labels. Exemplary labels include, but are not limited to, fluorophores, radioisotopes, Quantum Dots, chromogens, enzymes, antigens including but not limited to epitope tags, heavy metals, dyes, phosphorescence groups, chemiluminescent groups, electrochemical detection moieties, affinity tags, binding proteins, phosphors, rare earth chelates, near-infrared dyes, including but not limited to, “Cy.7.SPh.NCS,” “Cy.7.OphEt.NCS,” “Cy7.OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Nebr.), electrochemiluminescence labels, including but not limited to, tris(bipyridal) ruthenium (II), also known as Ru(bpy)₃²⁺, Os(1,10-phenanthroline)₂bis(diphenylphosphino)ethane²⁺, also known as Os(phen)₂(dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal) ruthenium (II), also known as Ru(v-bpy₃²⁺), and the like. Detailed descriptions of ECL and electrochemiluminescent moieties can be found in, among other places, A. Bard and L. Faulkner, Electrochemical Methods, John Wiley & Sons (2001); M. Collinson and M. Wightman, Anal. Chem. 65:2576 et seq. (1993); D. Brunce and M. Richter, Anal. Chem. 74:3157 et seq. (2002); A. Knight, Trends in Anal. Chem. 18:47 et seq. (1999); B. Muegge et al., Anal. Chem. 75:1102 et seq. (2003); H. Abrunda et al., J. Amer. Chem. Soc. 104:2641 et seq. (1982); K. Maness et al., J. Amer. Chem. Soc. 118:10609 et seq. (1996); M. Collinson and R. Wightman, Science 268:1883 et seq. (1995); and U.S. Pat. No. 6,479,233.

As used herein, the term “fluorophore” refers to a label that comprises a resonance-delocalized system or aromatic ring system that absorbs light at a first wavelength and emits fluorescent light at a second wavelength in response to the absorption event. A wide variety of such dye molecules are known in the art. For example, fluorescent dyes can be selected from any of a variety of classes of fluorescent compounds, such as xanthenes, rhodamines, fluoresceins, cyanines, phthalocyanines, squaraines, and bodipy dyes. In some embodiments, the dye comprises a xanthene-type dye, which contains a fused three-ring system of the form: 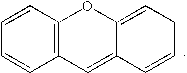
This parent xanthene ring may be unsubstituted (i.e., all substituents are H) or can be substituted with one or more of a variety of the same or different substituents, such as described below. In some embodiments, the dye contains a parent xanthene ring having the general structure: 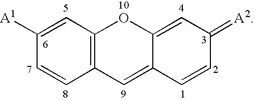
In the parent xanthene ring depicted above, A¹ is OH or NH₂ and A² is O or NH₂⁺. When A¹ is OH and A² is 0, the parent xanthene ring is a fluorescein-type xanthene ring. When A¹ is NH₂ and A² is NH₂⁺, the parent xanthene ring is a rhodamine-type xanthene ring. When A¹ is NH₂ and A² is O, the parent xanthene ring is a rhodol-type xanthene ring. In the parent xanthene ring depicted above, one or both nitrogens of A¹ and A² (when present) and/or one or more of the carbon atoms at positions C1, C2, C4, C5, C7, C8 and C9 can be independently substituted with a wide variety of the same or different substituents. In some embodiments, typical substituents can include, but are not limited to, —X, —R, —OR, —SR, —NRR, perhalo (C₁-C₆) alkyl, —CX₃, —CF₃, —CN, —OCN, —SCN, —NCO, —NCS, —NO, —NO₂, —N₃, —S(O)₂O⁻, —S(O)₂OH, —S(O)₂R, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F or Cl) and each R is independently hydrogen, (C₁-C₆) alkyl, (C₁-C₆) alkanyl, (C₁-C₆) alkenyl, (C₁-C₆) alkynyl, (C₅-C₂₀) aryl, (C₆-C₂₆) arylalkyl, (C₅-C₂₀) arylaryl, heteroaryl, 6-26 membered heteroarylalkyl 5-20 membered heteroaryl-heteroaryl, carboxyl, acetyl, sulfonyl, sulfinyl, sulfone, phosphate, or phosphonate. Moreover, the C1 and C2 substituents and/or the C7 and C8 substituents can be taken together to form substituted or unsubstituted buta[1,3]dieno or (C₅-C₂₀) aryleno bridges. Generally, substituents that do not tend to quench the fluorescence of the parent xanthene ring are preferred, but in some embodiments quenching substituents may be desirable. Substituents that tend to quench fluorescence of parent xanthene rings are electron-withdrawing groups, such as —NO₂, —Br, and —I. In some embodiments, C9 is unsubstituted. In some embodiments, C9 is substituted with a phenyl group. In some embodiments, C9 is substituted with a substituent other than phenyl. When A¹ is NH₂ and/or A² is NH₂⁺, these nitrogens can be included in one or more bridges involving the same nitrogen atom or adjacent carbon atoms, e.g., (C₁-C₁₂) alkyldiyl, (C₁-C₁₂) alkyleno, 2-12 membered heteroalkyldiyl and/or 2-12 membered heteroalkyleno bridges. Any of the substituents on carbons C1, C2, C4, C5, C7, C8, C9 and/or nitrogen atoms at C3 and/or C6 (when present) can be further substituted with one or more of the same or different substituents, which are typically selected from —X, —R′, ═O, —OR′, —SR′, ═S, —NR′R′, ═NR′, —CX₃, —CN, —OCN, —SCN, —NCO, —NCS, —NO, —NO₂, ═N₂, —N₃, —NHOH, —S(O)₂O⁻, —S(O)₂OH, —S(O)₂R′, —P(O)(O—)₂, —P(O)(OH)₂, —C(O)R′, —C(O)X, —C(S)R′, —C(S)X, —C(O)OR′, —C(O)O—, —C(S)OR′, —C(O)SR′, —C(S)SR′, —C(O)NR′R′, —C(S)NR′R′ and —C(NR)NR′R′, where each X is independently a halogen (preferably —F or —Cl) and each R′ is independently hydrogen, (C₁-C₆) alkyl, 2-6 membered heteroalkyl, (C₅-C₁₄) aryl or heteroaryl, carboxyl, acetyl, sulfonyl, sulfinyl, sulfone, phosphate, or phosphonate.

Exemplary parent xanthene rings include, but are not limited to, rhodamine-type parent xanthene rings and fluorescein-type parent xanthene rings.

In one embodiment, the dye contains a rhodamine-type xanthene dye that includes the following ring system: 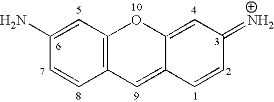
In the rhodamine-type xanthene ring depicted above, one or both nitrogens and/or one or more of the carbons at positions C1, C2, C4, C5, C7 or C8 can be independently substituted with a wide variety of the same or different substituents, as described above for the parent xanthene rings, for example. C9 may be substituted with hydrogen or other substituent, such as an orthocarboxyphenyl or ortho(sulfonic acid)phenyl group. Exemplary rhodamine-type xanthene dyes can include, but are not limited to, the xanthene rings of the rhodamine dyes described in U.S. Pat. Nos. 5,936,087, 5,750,409, 5,366,860, 5,231,191, 5,840,999, 5,847,162, and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483 (1992). Also included within the definition of “rhodamine-type xanthene ring” are the extended-conjugation xanthene rings of the extended rhodamine dyes described in U.S. application Ser. No. 09/325,243 filed Jun. 3, 1999.

In some embodiments, the dye comprises a fluorescein-type parent xanthene ring having the structure: 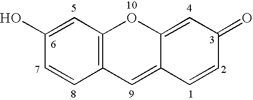
In the fluorescein-type parent xanthene ring depicted above, one or more of the carbons at positions C1, C2, C4, C5, C7, C8 and C9 can be independently substituted with a wide variety of the same or different substituents, as described above for the parent xanthene rings. C9 may be substituted with hydrogen or other substituent, such as an orthocarboxyphenyl or ortho(sulfonic acid)phenyl group. Exemplary fluorescein-type parent xanthene rings include, but are not limited to, the xanthene rings of the fluorescein dyes described in U.S. Pat. Nos. 4,439,356, 4,481,136, 4,933,471 (Lee), U.S. Pat. No. 5,066,580 (Lee), U.S. Pat. Nos. 5,188,934, 5,654,442, and 5,840,999, WO 99/16832, and EP 050684. Also included within the definition of “fluorescein-type parent xanthene ring” are the extended xanthene rings of the fluorescein dyes described in U.S. Pat. Nos. 5,750,409 and 5,066,580. In some embodiments, the dye comprises a rhodamine dye, which can comprise a rhodamine-type xanthene ring in which the C9 carbon atom is substituted with an orthocarboxy phenyl substituent (pendent phenyl group). Such compounds are also referred to herein as orthocarboxyfluoresceins. In some embodiments, a subset of rhodamine dyes are 4,7,-dichlororhodamines. Typical rhodamine dyes can include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional rhodamine dyes can be found, for example, in U.S. Pat. No. 5,366,860 (Bergot et al.), U.S. Pat. No. 5,847,162 (Lee et al.), U.S. Pat. No. 6,017,712 (Lee et al.), U.S. Pat. No. 6,025,505 (Lee et al.), U.S. Pat. No. 6,080,852 (Lee et al.), U.S. Pat. No. 5,936,087 (Benson et al.), U.S. Pat. No. 6,111,116 (Benson et al.), U.S. Pat. No. 6,051,719 (Benson et al.), U.S. Pat. Nos. 5,750,409, 5,366,860, 5,231,191, 5,840,999, and 5,847,162, U.S. Pat. No. 6,248,884 (Lam et al.), PCT Publications WO 97/36960 and WO 99/27020, Sauer et al., 1995, J. Fluorescence 5(3):247-261, Arden-Jacob, 1993, Neue Lanwellige Xanthen-Farbstoffe für Fluoresenzsonden und Farbstoff Laser, Verlag Shaker, Germany, and Lee et al., Nucl. Acids Res. 20(10):2471-2483 (1992), Lee et al., Nucl. Acids Res. 25:2816-2822 (1997), and Rosenblum et al., Nucl. Acids Res. 25:4500-4504 (1997), for example. In some embodiments, the dye comprises a 4,7-dichloro-orthocarboxyrhodamine. In some embodiments, the dye comprises a fluorescein dye, which comprises a fluorescein-type xanthene ring in which the C9 carbon atom is substituted with an orthocarboxy phenyl substituent (pendent phenyl group). One typical subset of fluorescein-type dyes are 4,7,-dichlorofluoresceins. Typical fluorescein dyes can include, but are not limited to, 5-carboxyfluorescein (5-FAM), 6-carboxyfluorescein (6-FAM). Additional typical fluorescein dyes can be found, for example, in U.S. Pat. Nos. 5,750,409, 5,066,580, 4,439,356, 4,481,136, 4,933,471 (Lee), 5,066,580 (Lee), 5,188,934 (Menchen et al.), 5,654,442 (Menchen et al.), 6,008,379 (Benson et al.), and 5,840,999, PCT publication WO 99/16832, and EPO Publication 050684. In some embodiments, the dye comprises a 4,7-dichloro-orthocarboxyfluorescein. In some embodiments, the dye can be a cyanine, phthalocyanine, squaraine, or bodipy dye, such as described in the following references and references cited therein: U.S. Pat. No. 5,863,727 (Lee et al.), U.S. Pat. No. 5,800,996 (Lee et al.), U.S. Pat. No. 5,945,526 (Lee et al.), U.S. Pat. No. 6,080,868 (Lee et al.), U.S. Pat. No. 5,436,134 (Haugland et al.), U.S. Pat. No. 5,863,753 (Haugland et al.), U.S. Pat. No. 6,005,113 (Wu et al.), and WO 96/04405 (Glazer et al.).

As used herein, the term “target identifying portion” refers to a moiety or moieties that can be used to identify a particular probe species and target polynucleotide, and can refer to a variety of distinguishable moieties, including for example zipcodes and a known number of nucleobases. In some embodiments, identifying portion refers to an oligonucleotide sequence that can be used for separating the element to which it is bound, including without limitation, bulk separation; for tethering or attaching the element to which it is bound to a substrate, which may or may not include separating; for annealing an identifying portion complement that may comprise at least one moiety, such as a mobility modifier, one or more labels, and combinations thereof. The term “target identifying portion complement” typically refers to at least one oligonucleotide that comprises at least one sequence of nucleobases that are at least substantially complementary to and hybridize with their corresponding identifying portion. In some embodiments, at least one identifying portion complement comprises at least one reporter group and serves as a label for at least one ligation product, at least one ligation product surrogate, and combinations thereof. In some embodiments, determining comprises detecting one or more reporter groups on at least one identifying portion complement.

Typically, target identifying portions and their corresponding target identifying portion complements are selected to minimize: internal, self-hybridization; cross-hybridization with different target identifying portion species, nucleotide sequences in a reaction composition, including but not limited to gDNA, different species of target identifying portion complements, or target-specific portions of probes, and the like; but should be amenable to facile hybridization between the target identifying portion and its corresponding target identifying portion complement. Target identifying portion sequences and target identifying portion complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65 (1998)). Descriptions of target identifying portions can be found in, among other places, U.S. Pat. No. 6,309,829 (referred to as “tag segment” therein); 6,451,525 (referred to as “tag segment” therein); 6,309,829 (referred to as “tag segment” therein); 5,981,176 (referred to as “grid oligonucleotides” therein); 5,935,793 (referred to as “identifier tags” therein); and PCT Publication No. WO 01/92579 (referred to as “addressable support-specific sequences” therein).

In some embodiments, target identifying portions are at least 12 bases in length, at least 15 bases in length, 12-60 bases in length, or 15-30 bases in length. In some embodiments, at least one identifying portion is 12, 15, 20, 21, 22, 23, 24, 25, 26, 27, 28, 29, 30, 45, or 60 bases in length. In some embodiments, at least two identifying portion: identifying portion complement duplexes have melting temperatures that fall within a Δ Tₘ range (Tₘₐₓ-Tₘᵢₙ) of no more than 10° C. of each other. In some embodiments, at least two identifying portion: identifying portion complement duplexes have melting temperatures that fall within a Δ Tₘ range of 5° C. or less of each other. In some embodiments, at least two identifying portion: identifying portion complement duplexes have melting temperatures that fall within a Δ Tₘ range of 2° C. or less of each other. In some embodiments, at least one target identifying portion complement is annealed to at least one corresponding target identifying portion and, subsequently, at least part of that identifying portion complement is released and detected.

The term “mobility modifier” as used herein refers to at least one molecular entity, for example but not limited to, at least one polymer chain, that when added to at least one element (e.g., at least one probe, at least one primer, at least one ligation product, at least one ligation product surrogate, at least one mobility probe, or combinations thereof) affects the mobility of the element to which it is hybridized or bound, covalently or non-covalently, in at least one mobility-dependent analytical technique. Typically, a mobility modifier changes the charge/translational frictional drag when hybridized or bound to the element; or imparts a distinctive mobility, for example but not limited to, a distinctive elution characteristic in a chromatographic separation medium or a distinctive electrophoretic mobility in a sieving matrix or non-sieving matrix, when hybridized or bound to the corresponding element; or both (see, e.g., U.S. Pat. Nos. 5,470,705 and 5,514,543). In some embodiments, a multiplicity of probes exclusive of mobility modifiers, a multiplicity of primers exclusive of mobility modifiers, a multiplicity of ligation products exclusive of mobility modifiers, a multiplicity of ligation product surrogates exclusive of mobility modifiers, or combinations thereof, have the same or substantially the same mobility in at least one mobility-dependent analytical technique. For various examples of mobility modifiers see for example U.S. Pat. Nos. 6,395,486, 6,358,385, 6,355,709, 5,916,426, 5,807,682, 5,777,096, 5,703,222, 5,556,7292, 5,567,292, 5,552,028, 5,470,705, and Barbier et al., Current Opinion in Biotechnology, 2003,14:1:51-57

In some embodiments, at least one mobility modifier comprises at least one nucleotide polymer chain, including without limitation, at least one oligonucleotide polymer chain, at least one polynucleotide polymer chain, or both at least one oligonucleotide polymer chain and at least one polynucleotide polymer chain (see for example Published P.C.T. application WO9615271 A1, as well as product literature for Keygene SNPWave™ for some examples of using known numbers of nucleotides to confer mobility to ligation products). In some embodiments, at least one mobility modifier comprises at least one non-nucleotide polymer chain. Exemplary non-nucleotide polymer chains include, without limitation, peptides, polypeptides, polyethylene oxide (PEO), or the like. In some embodiments, at least one polymer chain comprises at least one substantially uncharged, water-soluble chain, such as a chain composed of PEO units; a polypeptide chain; or combinations thereof.

The polymer chain can comprise a homopolymer, a random copolymer, a block copolymer, or combinations thereof. Furthermore, the polymer chain can have a linear architecture, a comb architecture, a branched architecture, a dendritic architecture (e.g., polymers containing polyamidoamine branched polymers, Polysciences, Inc. Warrington, Pa.), or combinations thereof. In some embodiments, at least one polymer chain is hydrophilic, or at least sufficiently hydrophilic when hybridized or bound to an element to ensure that the element-mobility modifier is readily soluble in aqueous medium. Where the mobility-dependent analysis technique is electrophoresis, in some embodiments, the polymer chains are uncharged or have a charge/subunit density that is substantially less than that of its corresponding element.

The synthesis of polymer chains useful as mobility modifiers will depend, at least in part, on the nature of the polymer. Methods for preparing suitable polymers generally follow well-known polymer subunit synthesis methods. These methods, which involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, oligosaccharides, and nucleotide polymers. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Int. J. Peptide Protein Res., 35: 161-214 (1990)).

One method for preparing PEO polymer chains having a selected number of hexaethylene oxide (HEO) units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., U.S. Pat. No. 4,914,210; see also, U.S. Pat. No. 5,777,096).

As used herein, a “mobility probe” generally refers to a molecule comprising a mobility modifier, a label, and a target identifying portion or target identifying portion complement that can hybridize to a ligation product or ligation product surrogate, the detection of which allows for the identification of the target polynucleotide.

As used herein, the term “mobility-dependent analytical technique” as used herein refers to any means for separating different molecular species based on differential rates of migration of those different molecular species in one or more separation techniques. Exemplary mobility-dependent analysis techniques include gel electrophoresis, capillary electrophoresis, chromatography, capillary electrochromatography, mass spectroscopy, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682, PCT Publication No. WO 01/92579, Fu et al., Current Opinion in Biotechnology, 2003, 14:1:96-100, D. R. Baker, Capillary Electrophoresis, Wiley-Interscience (1995), Biochromatography: Theory and Practice, M. A. Vijayalakshmi, ed., Taylor & Francis, London, U.K. (2003); and A. Pingoud et al., Biochemical Methods: A Concise Guide for Students and Researchers, Wiley-VCH Verlag GmbH, Weinheim, Germany (2002).

As used herein, the term “ligation agent”, according to the present invention, can comprise any number of enzymatic or non-enzymatic reagents. For example, ligase is an enzymatic ligation reagent that, under appropriate conditions, forms phosphodiester bonds between the 3′-OH and the 5′-phosphate of adjacent nucleotides in DNA molecules, RNA molecules, or hybrids. Temperature sensitive ligases, include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase (see for example Published P.C.T. Application WO00/26381, Wu et al., Gene, 76(2):245-254, (1989), Luo et al., Nucleic Acids Research, 24(15): 3071-3078 (1996). The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits. Further, reversibly inactivated enzymes (see for example U.S. Pat. No. 5,773,258) can be employed in some embodiments of the present teachings.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

Photoligation using light of an appropriate wavelength as a ligation agent is also within the scope of the teachings. In some embodiments, photoligation comprises probes comprising nucleotide analogs, including but not limited to, 4-thiothymidine (s⁴T), 5-vinyluracil and its derivatives, or combinations thereof. In some embodiments, the ligation agent comprises: (a) light in the UV-A range (about 320 nm to about 400 nm), the UV-B range (about 290 nm to about 320 nm), or combinations thereof, (b) light with a wavelength between about 300 nm and about 375 nm, (c) light with a wavelength of about 360 nm to about 370 nm; (d) light with a wavelength of about 364 nm to about 368 nm, or (e) light with a wavelength of about 366 nm. In some embodiments, photoligation is reversible. Descriptions of photoligation can be found in, among other places, Fujimoto et al., Nucl. Acid Symp. Ser. 42:39-40 (1999); Fujimoto et al., Nucl. Acid Res. Suppl. 1:185-86 (2001); Fujimoto et al., Nucl. Acid Suppl., 2:155-56 (2002); Liu and Taylor, Nucl. Acid Res. 26:3300-04 (1998) and on the world wide web at: sbchem.kyoto-u.ac.jp/saito-lab.

Ligation

Ligation according to the present teachings comprises any enzymatic or non-enzymatic process wherein an inter-nucleotide linkage is formed between the opposing ends of nucleic acid sequences that are adjacently hybridized to a template. Typically, the opposing ends of the annealed nucleic acid probes are suitable for ligation (suitability for ligation is a function of the ligation means employed). In some embodiments, ligation also comprises at least one gap-filling procedure, wherein the ends of the two probes are adjacent but not contiguoulsy hybridized initially, but the 3′-end of the first probe is extended by one or more nucleotide until it is contiguous to the 5′-end of the second probe, typically by a polymerase (see, e.g., U.S. Pat. No. 6,004,826). The internucleotide linkage can include, but is not limited to, phosphodiester bond formation. Such bond formation can include, without limitation, those created enzymatically by at least one DNA ligase or at least one RNA ligase, for example but not limited to, T4 DNA ligase, T4 RNA ligase, Thermus thermophilus (Tth) ligase, Thermus aquaticus (Taq) DNA ligase, Thermus scotoductus (Tsc) ligase, TS2126 (a thermophilic phage that infects Tsc) RNA ligase, Archaeoglobus flugidus (Afu) ligase, Pyrococcus furiosus (Pfu) ligase, or the like, including but not limited to reversibly inactivated ligases (see, e.g., U.S. Pat. No. 5,773,258), and enzymatically active mutants and variants thereof.

Other internucleotide linkages include, without limitation, covalent bond formation between appropriate reactive groups such as between an α-haloacyl group and a phosphothioate group to form a thiophosphorylacetylamino group, a phosphorothioate a tosylate or iodide group to form a 5′-phosphorothioester, and pyrophosphate linkages.

Chemical ligation can, under appropriate conditions, occur spontaneously such as by autoligation. Alternatively, “activating” or reducing agents can be used. Examples of activating and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light, such as used for photoligation.

Ligation generally comprises at least one cycle of ligation, i.e., the sequential procedures of: hybridizing the target-specific portions of a first probe and a corresponding second probe to their respective complementary regions on the corresponding target nucleic acid sequences; ligating the 3′ end of the first probe with the 5′ end of the second probe to form a ligation product; and denaturing the nucleic acid duplex to release the ligation product from the ligation product:target nucleic acid sequence duplex. The ligation cycle may or may not be repeated, for example, without limitation, by thermocycling the ligation reaction to amplify the ligation product using ligation probes (as distinct from using primers and polymerase to generate amplified ligation products).

Also within the scope of the teachings are ligation techniques such as gap-filling ligation, including, without limitation, gap-filling versions OLA, LDR, LCR, FEN-cleavage mediated versions of OLA, LDR, and LCR, bridging oligonucleotide ligation, correction ligation, and looped linker-based concatameric ligation. Additional non-limiting ligation techniques included within the present teachings comprise OLA followed by PCR (see for example Rosemblum et al, P.C.T. Application US03/37227, Rosemblum et al., P.C.T. Application US03/37212 and Barany et al., Published P.C.T. application WO974559A1, OLA comprising mobility modifiers (see for example U.S. Pat. No. 5,514,543, PCR followed by OLA, two PCR's followed by an OLA, ligation comprising single circularizable probes (see for example Landregren et al., WO9741254A1, OLA comprising rolling circle replication of padlock probes (see for example Landregren et al., U.S. Pat. No. 6,558,928. Additional descriptions of these and related techniques can be found in, among other places, U.S. Pat. Nos. 5,185,243 and 6,004,826, 5,830,711, 6,511,810, 6,027,889; published European Patent Applications EP 320308 and EP 439182; Published PCT applications WO 90/01069, WO 01/57268, WO0056927A3, WO9803673A1, WO200117329, Landegren et al., Science 241:1077-80 (1988), Day et al., Genomics, 29(1): 152-162 (1995), de Arruda et al., and U.S. Application No. 60/517470.

Methods for removing unhybridized and/or unligated probes following a ligation reaction are known in the art, and are further discussed infra. Such procedures include nuclease-mediated approaches, dilution, size exclusion approaches, affinity moiety procedures, (see for example U.S. Provisional Application 60/517,470, U.S. Provisional Application 60/477,614, and P.C.T. Application 2003/37227), affinity-moiety procedures involving immobilization of target polynucleotides (see for example Published P.C.T. Application WO 03/006677A2).

Amplification

Amplification according to the present teachings encompass any manner by which at least a part of at least one target polynucleotide is reproduced, typically in a template-dependent manner, including without limitation, a broad range of techniques for amplifying nucleic acid sequences, either linearly or exponentially. Exemplary steps for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA) and the like, including multiplex versions and combinations thereof. Descriptions of such techniques can be found in, among other places, Sambrook and Russell; Sambrook et al.; Ausbel et al.; PCR Primer: A Laboratory Manual, Diffenbach, Ed., Cold Spring Harbor Press (1995); The Electronic Protocol Book, Chang Bioscience (2002)(“The Electronic Protocol Book”); Msuih et al., J. Clin. Micro. 34:501-07 (1996); The Nucleic Acid Protocols Handbook, R. Rapley, ed., Humana Press, Totowa, N.J. (2002)(“Rapley”); Abramson et al., Curr Opin Biotechnol. 1993 February; 4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May;53(2):165-74, Schweitzer et al., Curr Opin Biotechnol. 2001 February; 12(1):21-7, U.S. Pat. No. 5,830,711, U.S. Pat. No. 6,027,889, U.S. Pat. No. 5,686,243, Published P.C.T. Application WO0056927A3, and Published P.C.T. Application WO9803673A1. Amplification can comprise thermocycling or can be performed isothermally. In some embodiments, newly-formed nucleic acid duplexes are not initially denatured, but are used in their double-stranded form in one or more subsequent steps.

Primer extension is an amplifying step that comprises elongating at least one probe or at least one primer that is annealed to a template in the 5′ to 3′ direction using an amplifying means such as a polymerase. According to some embodiments, with appropriate buffers, salts, pH, temperature, and nucleotide triphosphates, including analogs thereof, i.e., under appropriate conditions, a polymerase incorporates nucleotides complementary to the template strand starting at the 3′-end of an annealed probe or primer, to generate a complementary strand. In some embodiments, primer extension can be used to fill a gap between two probes of a probe set that are hybridized to target sequences of at least one target nucleic acid sequence so that the two probes can be ligated together. In some embodiments, the polymerase used for primer extension lacks or substantially lacks 5′ exonuclease activity.

In some embodiments of the present teachings, unconventional nucleotide bases can be introduced into the amplification reaction products and the products treated by enzymatic (e.g., glycosylases) and/or physical-chemical means in order to render the product incapable of acting as a template for subsequent amplifications. In some embodiments, uracil can be included as a nucleobase in the reaction mixture, thereby allowing for subsequent reactions to decontaminate carrover of previous uracil-containing products by the use of uracil-N-glycosylase (see for example Published P.C.T. Application WO9201814A2). In some embodiments of the present teachings, any of a variety of techniques can be employed prior to amplification in order to facilitate amplification success, as described for example in Radstrom et al., Mol Biotechnol. 2004 February; 26(2):13346. In some embodiments, amplification can be achieved in a self-contained integrated approach comprising sample preparation and detection, as described for example in U.S. Pat. Nos. 6,153,425 and 6,649,378.

Detection and Quantification

Detection and quantification can be carried out using a variety of procedures, including for example mobility dependent analysis techniques (for example capillary or gel electrophoresis), solid support comprising array capture oligonucleotides, various bead approaches (see for example Published P.C.T. Application WO US02/37499), including fiber optics, as well as flow cytometry (for example, FACS).

The use of capillary and gel electrophoresis for detection and quantification of target polynucleotides is well known, see for example, Grossman, et al., “High-density Multiplex Detection of Nucleic Acid Sequences: Oligonucleotide Ligation Assay and Sequence-coded Separation,” Nucl. Acids Res. 22(21): 4527-34 (1994), Slater et al., Current Opinion in Biotechnology, 2003, 14:1:58-64, product literature for the Applied Biosystems 3100, 3700, and 3730 capillary electrophoresis instruments, and product literature for the SNPlex Genotyping System Chemistry Guide, also from Applied Biosystems. Additional mobility dependent analysis techniques that can provide for detection and quantification according to the present teachings include mass spectroscopy (optionally comprising a deconvolution step via chromatography), collision-induced dissociation (CID) fragmentation analysis, fast atomic bombardment and plasma desorption, and electrospray/ionspray (ES) and matrix-assisted laser desorption/ionization (MALDI) mass spectrometry. In some embodiments, MALDI mass spectrometry can be used with a time-of-flight (TOF) configuration (MALDI-TOF, see for example Published P.C.T. Application WO 97/33000), and MALDI-TOF-TOF (see for example Applied Biosystems 4700 Proteomics Discovery System product literature). Additional mass spectrometry approaches for detection and quantification are described for example in the Applied Biosystems Qtrap LC/MS/MS System product literature, the Applied Biosystems QSTAR XL Hybrid LC/MS/MS System product literature, the Applied Biosystems Q TRAP™ LC/MS/MS System product literature, and the Applied Biosystems Voyager-DE™ PRO Biospectrometry Workstation product literature.

In some embodiments of the present teachings, analysis of detected products can be undertaken with the application of various software procedures. For example, analysis of capillary electrophoresis products can employ various commercially available software packages from Applied Biosystems, for example GeneMapper version 3.5 and BioTrekker version 1.0.

In general, it will be appreciated that the process employed for detection and quantification is not a limitation of the present teachings

Aspects of the present teachings may be further understood in light of the following examples, which should not be construed as limiting the scope of the teachings in any way.

### Exemplary Embodiments

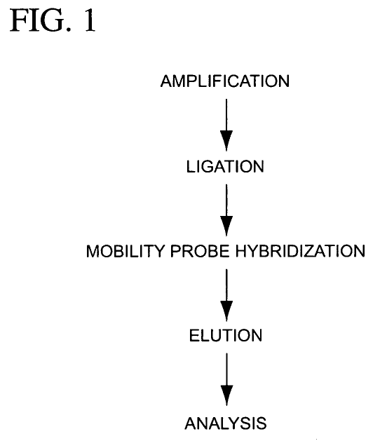

In some embodiments of the present teachings as depicted in FIG. 1, an amplification reaction is performed, followed by a ligation reaction. Thereafter, mobility probes are hybridized to the ligation products. Determination of the target polynucleotide sequence results from analysis of the eluted bound mobility probes

In some embodiments of the present teachings, a target polynucleotide sequence is detected. For example, as depicted in FIG. 2, a target polynucleotide sequence comprising a T:A base pair can be amplified with a pair of flanking primers in an amplification reaction (here, a PCR amplification reaction, though it will be appreciated that a variety of amplification reactions can be employed within the context of the present teachings). After the amplification reaction, a ligation reaction is performed, wherein the amplification product is queried with a ligation probe set comprising a first probe and a second probe. Here, the first probe comprises a target specific portion (TSP) complementary to the amplified target polynucleotide sequence, and a target identifying portion (TIP). The second probe comprises a target specific portion, and an affinity moiety (here, a biotin molecule, though it will be appreciated that a variety of affinity moieties can be employed within the context of the present teachings). Also depicted in FIG. 1 is a single ligation reaction, though it will be appreciated that various cycling ligation reactions are within the scope of the present teaching (for example, the ligase detection reaction (LDR) (see reference), the Ligase Chain Reaction (LCR) (see reference), gap-filling version of LDR and LCR (see reference), cleavage/ligation reactions such as FEN-LCR (see Bi), as well as others. After the ligation reaction, the ligation products are contacted with an affinity moiety binder(here streptavidin, though it will be appreciated that a variety of affinity moiety binders can be employed within the context of the present teachings). Unbound molecules can then be removed. Mobility probes can then be hybridized to the bound affinity-moiety strands, where the mobility probe comprises a CTIP region that is complementary to the TIP of the immobilized ligation product. Unhybridized Mobility probes are then washed away. The remaining Mobility probes can then be eluted, and analyzed using a mobility dependent analysis technique.

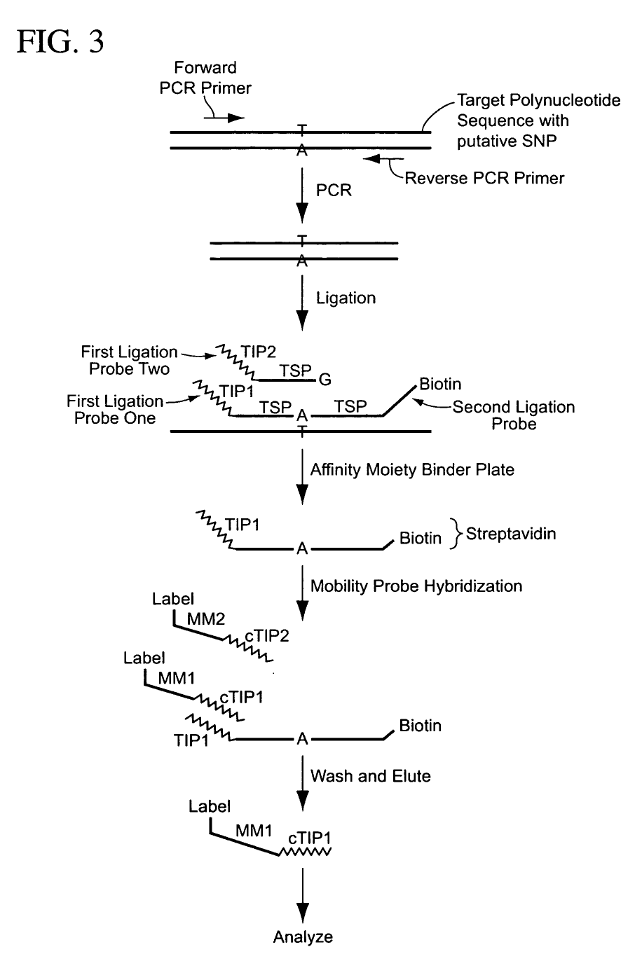

In some embodiments of the present teachings, a target polynucleotide sequence comprising a putative SNP is detected. For example, as depicted in FIG. 3, a target polynucleotide sequence comprising a T:A base pair at a putative SNP locus can be amplified with a pair of flanking primers in an amplification reaction. After the amplification reaction, a ligation reaction is performed, wherein the amplification product is queried with a ligation probe set comprising a first probe and a second probe. Here, a first probe one comprises a target specific portion (TSP) complementary to the amplified target polynucleotide sequence, a discriminating nucleotide at its 3′ end (here, an A), and a target identifying portion one (TIP1). A first probe two comprises a target specific portion (TSP) complementary to the amplified target polynucleotide sequence, a discriminating nucleotide at its 3′ end (here, a G), and a target identifying portion two (TIP2). The second probe comprises a target specific portion, and an affinity moiety (here, a biotin molecule, though it will be appreciated that a variety of affinity moieties can be employed within the context of the present teachings). After the ligation reaction, the ligation products are contacted with an affinity moiety binder. Unbound molecules can then be removed. Mobility probes can then be hybridized to the bound affinity-moiety strands, where the mobility probe comprises a cTIP region that is complementary to the TIP of the immobilized ligation product. Here, the Mobility probe comprising the cTIP1 hybridizes to the bound ligation product comprising TIP1, whereas the Mobility probe comprising cTIP2 does not have a corresponding bound ligation product comprising a TIP2, and is thus washed during the wash. The remaining hybridized Mobility probes can then be eluted, and analyzed using a mobility dependent analysis technique.

In some embodiments, mobility probes corresponding to the target identifying portions of different ligation products are distinguished from one another based on the distinct mobility conferred by their mobility modifying portion. In some embodiments, Mobility probes corresponding to the target identifying portions of different ligation products are distinguishable from one another based on a distinct label. In some embodiments, the mobility probes corresponding to alternate alleles of a given SNP locus comprises the same mobility modifying portion but different labels. In some embodiments, the mobility probes corresponding to alternate alleles of a given SNP locus comprise different mobility modifying portions but the same labels. In some embodiments, the mobility probes corresponding to alternate alleles of a given SNP locus comprises different, but adjacently eluting mobility modifying portions (that is, they elute next to each other in an electropherrogram resulting from capillary electrophoresis, for example), but different labels. In some embodiments, the mobility probes corresponding to alternate alleles of a given SNP locus comprises different, but adjacently eluting mobility modifying portions (that is, they elute next to each other in an electropherrogram resulting from capillary electrophoresis, for example), and the same labels. Generally, it will appreciated that one having ordinary skill in the art can employ a variety of ways of coding the identity of a particular target polynucleotide sequence with the mobility modifying portion and the label portion of the mobility probes, and that such procedures are routine and do not require undue experimentation.

In some embodiments of the present teachings, a plurality of target polynucleotide sequences are amplified in a multiplexed amplification reaction. In some embodiments, a plurality of target polynucleotide sequences are amplified in a multiplexed amplification reaction, wherein the plurality of target polynucleotide sequences are all derived from the same gene. In some embodiments, the gene is human ACE. In some embodiments, the gene is human ApoB. In some embodiments, the gene is human Apo CIII. In some embodiments, the gene is human Factor V. In some embodiments, the gene is human MTHFR. In some embodiments, the gene is the human cystic fibrosis (CFTR) gene. In some embodiments, the gene is the human Familial Hypercholesterolemia (LDL receptor) gene. It will be appreciated that the particular target polynucleotide sequence queried is not a limitation of the present teachings.

In some embodiments, a single target polynucleotide is queried according to the amplification and ligation strategies of the present teachings. In some embodiments, a plurality of target polynucleotide is queried according to the amplification and ligation strategies of the present teachings. In some embodiments, the plurality of target polynucleotide queried according to the amplification and ligation strategies of the present teachings comprises at least 10 target polynucleotide sequences. In some embodiments, the plurality of target polynucleotide queried according to the amplification and ligation strategies of the present teachings comprises at least 20 target polynucleotide sequences. In some embodiments, the plurality of target polynucleotide queried according to the amplification and ligation strategies of the present teachings comprises at least 30 target polynucleotide sequences. In some embodiments, the plurality of target polynucleotide queried according to the amplification and ligation strategies of the present teachings comprises at least 40 target polynucleotide sequences. In some embodiments, the plurality of target polynucleotide queried according to the amplification and ligation strategies of the present teachings comprises at least 50 target polynucleotide sequences. In some embodiments, the plurality of target polynucleotide queried according to the amplification and ligation strategies of the present teachings comprises at least 100 target polynucleotide sequences. In some embodiments, the plurality of target polynucleotide queried according to the amplification and ligation strategies of the present teachings comprises at least 200 target polynucleotide sequences.

It will be appreciated that the nature of the target polynuclotide sequence queried is not a limitation of the present teachings, and can include for example genomic DNA (corresponding to both introns and exons), expressed RNA (mRNA), other forms of RNA including micro RNA, as well as any of a variety of other natural and non-natural nucleobase sequences. For example, some embodiments of the present teachings comprise strategies employing the ligation of linkers to micro RNA molecules. Following ligation of linkers to micro RNA, primer sequences introduced in the linkers can be used to amplify the micro RNA molecules. Following amplification, ligation probe sets of the present teachings can be employed to query the ligation product, and mobility probes of the present teachings employed to analyze the micro RNAs. For additional guidance on the use of linker compositions to analyze micro RNA molecules see U.S. Non-Provisional application Ser. No. 10/947,460, to Chen et al.,

In some embodiments, the target polynucleotide sequence queried can be the result of bisulfite treatment, and methylation status of cytosine determined as a result of cytosine conversion into thymine residues in an amplification reaction (see for example Popiela et al., 2004, Eur J Gynaecol Oncol. 25(2):145-9, Boyd et al., U.S. Provisional Application 60/499,082, Boyd et al., U.S. Non-Provisional patent application Ser. No. 10/926,531, and Fraga et al., Biotechniques. 2002 September; 33(3):632, 634, 636-49).

### EXAMPLE

### PCR Protocol

1) Amplify the target DNA with PCR mix, either singleplex or multiplex.
a) Add target DNA to PCR mix containing Taq Gold, dNTPs, buffer, MgCl2, primers (typically a 10-20 μL reaction volume).
b) Thermal cycle appropriately for the application.

2) Add PCR sample to ligation reaction mix containing:
a) 10× ligation buffer and ligase. Ligate with 40U total ligase.
b) ligation probe mix solution containing ligation probes.
c) Thermal cycle appropriate for the application (for example, 32×45 sec. Cycles)).
PCR sample volume added can be 10 μL for a 20 μL
OLA total reaction volume.

3) Remove 10 μL OLA mix and hybridize according to mobility probe hybridization protocol.

### Mobility Probe Hybridization Protocol

1. Wash a 96 or 384 well streptavidin plate w/100 μl 0.1×SSC/0.01% Tween-20 (3× wash)

2. Add 40 μL of 1×SSC/0.01% Tween-20 to the plate and 10 μL ligation product to the plate and mix to give a total volume of 50 μL in the plate. Cover the plate with a sticky plate cover and incubate for 60 min at Room Temperature.

3. Empty the plate and tamp on a paper towel. Wash the Streptavidin plate w/100 μL 0.1×SSC/0.01% Tween-20 three times.

4. Add 50 uL of 2.5 nM mobility probe mix (100 fold dilution) in 4×SSCPE.1% Tween-20. Cover the plate again with the sticky cover, and incubate the plate for 60 min at 50° C.

5. Empty the plate and tamp on a paper towel, and then wash the streptavidin plate four times using 100 μL 0.1×SSC/0.01% Tween-20.

6. Add 50 μL Hi-Di Formamide and cover the plate with the optical sticky plate cover, and incubate at 50° C. oven for 8 min. Spin briefly to recover any condensation. The samples can be transferred at this point to a fresh plate (e.g. a 96-well) at this point for storage.

7. Prepare a master mix of LIZ injection standard by adding 10 μL LIZ injection standard (commercially available as SNPlex™ Liz injection standard from Applied Biosystems) to 1000 μL SNPlex Injection Solution (also commercially available from Applied Biosystems). This prepares enough standard for 80 15 μL samples.

8. Dilute 5 uL of sample in 10 uL of SNPlex Injection Solution/SNPlex LIZ Internal Standard (40 uL SLIS in 1000 uL SNPlex Injection Solution). Inject into 3100 using a 36-cm array, POP6. Run at 980 seconds for electrophoresis time, determine empirically the injection conditions.

While the present teachings have been described in terms of these exemplary embodiments, the skilled artisan will readily understand that numerous variations and modifications of these exemplary embodiments are possible without undue experimentation. All such variations and modifications are within the scope of the current teachings.

We claim:

1. A method for analyzing a target polynucleotide sequence comprising;
providing a sample target polynucleotide sequence of interest;
amplifying a portion of the polynucleotide sequence of interest and producing an amplification product;
mixing the amplification product with a ligation probe set, the ligation probe set comprising a first ligation probe having a target specific portion complementary to a first strand of the amplicon, and a second ligation probe having a target specific portion complementary to the first strand of the amplicon, wherein the first ligation probe comprises a target identifying portion, wherein the second ligation probe comprises an affinity moiety, and wherein the ligation probes in a particular set are suitable for ligation together when hybridized adjacent to one another on the same strand of the amplification product;
ligating the first ligation probe to the second ligation probe to produce a ligation product, wherein the ligation product comprises (a) the target identifying portion, (b) the target specific portions ligated together, and (C) the affinity moiety;
contacting the ligation product with an affinity moiety binder to immobilize the ligation product;
hybridizing a mobility probe to the immobilized ligation product, wherein the mobility probe comprises a sequence complementary to the target identifying portion;
eluting the bound mobility probe;
detecting the eluted mobility probe, and;
analyzing the target polynucleotide sequence.

2. The method according to claim 1, further comprising,
providing a plurality of amplification primer pairs, each primer pair designed for amplifying a target polynucleotide sequences;
providing a plurality of ligation probe sets wherein each ligation probe set comprises a first ligation probe one, a first ligation probe two, and a second ligation probe, wherein the first ligation probe one hybridizes to a given allele in a base-specific manner, wherein first ligation probe two hybridizes to an alternate allele in a base-specific manner, and wherein the second ligation probe hybridizes adjacent to the first ligation probes, wherein the first ligation probe one and the first ligation probe two comprise distinguishing target identifying portions;
providing a plurality of mobility probes, wherein the mobility probes are complementary to a given target identifying portion;
hybridizing the mobility probes to the immobilized ligation products, and determining the identity of the target polynucleotide sequence.

3. The method according to claim 2 wherein the target identifying portion lacks substantial complementarity with sequences contained the plurality of target polynucleotide sequences.

4. The method according to claim 2 wherein the mobility dependent analysis technique is capillary electrophoresis.

5. The method according to claim 2 wherein the mobility probes comprise a label.

6. The method according to claim 5 wherein the label is selected from the group consisting of chromophores, fluorescent moieties, enzymes, antigens, heavy metals, magnetic probes, dyes, phosphorescent groups, radioactive materials, chemiluminescent moieties, and electrochemical detecting moieties.

7. The method according to claim 2 wherein the mobility probe comprises a mobility modifier.

8. The method according to claim 2 wherein the mobility probe comprises a label and a mobility modifier.

9. The method according to claim 2 wherein the affinity moiety is biotin.

10. The method according to claim 9 wherein the biotin is located on the 3′ end of the second ligation probe.

11. The method according to claim 2 wherein the affinity moiety binder is streptavidin.

12. The method according to claim 2, wherein the ligase reaction comprises a ligation detection reaction that comprises 2 to 65 cycles.

13. The method according to claim 2 wherein the ligase is selected from at least one of Thermus aquaticus ligase, Thermus thermophilus ligase, E. coli ligase, T4 ligase, AK16D, Pyrococcus ligase, and combinations thereof.

14. The method according to claim 2 wherein the amplification primer sets and ligation probe sets query a plurality of target polynucleotide sequences in the ACE gene.

15. The method according to claim 2 wherein the amplification primer sets and ligation probe sets query a plurality of target polynucleotide sequences in the ApoB gene.

16. The method according to claim 2 wherein the amplification primer sets and ligation probe sets query a plurality of target polynucleotide sequences in the Apo CIII gene.

17. The method according to claim 2 wherein the amplification primer sets and ligation probe sets query a plurality of target polynucleotide sequences in the Factor V gene.

18. The method according to claim 2 wherein the amplification primer sets and ligation probe sets query a plurality of target polynucleotide sequences in the MTHFR gene.

19. The method according to claim 2 wherein the amplification primer sets and ligation probe sets query a plurality of target polynucleotide sequences in the cystic fibrosis (CFTR) gene.

20. The method according to claim 2 wherein the amplification primer sets and ligation probe sets query a plurality of target polynucleotide sequences in the Familial Hypercholesterolemia (LDL receptor) gene.
